(19) United States
(12) Patent Application Publication
(10) Pub. No.: US 2009/0287064 A1
(43) Pub. Date: Nov. 19, 2009

(54) COMPUTER IMPLEMENTED COGNITIVE SELF TEST

(75) Inventors: John H. Dougherty, Jr., Lenoir City, TN (US); Andrew Dougherty, Knoxville, TN (US)

Correspondence Address: LUEDEKA, NEELY & GRAHAM, P.C., P O BOX 1871, KNOXVILLE, TN, 37901, US

(73) Assignee: MEDICAL INTERACTIVE EDUCATION, LLC, Lenoir City, TN (US)

(21) Appl. No.: 12/121,288

(22) Filed: May 15, 2008

(51) Int. Cl. A61B 5/00; G09B 19/00

(52) U.S. Cl. 600/300; 434/236

(57) ABSTRACT

A computer implemented method for cognitive testing, specifically for dementia-related afflictions. The method is administered on a computer workstation in an interactive, web-based format and screens the cognitive abilities of the patient. The method includes evaluation of five areas of cognitive domain including visuospatial skills and executive function by means of a clock face test, memory, verbal fluency, attention, and orientation, all of which are essential for indications of dementia, including Alzheimer's disease and other cognitive afflictions. The method does not require administration by a trained professional, but rather, is administered in a web browser either from a server to the workstation via the Internet or locally at the workstation.

### FIELD

This invention relates to the field of cognitive testing. More particularly, this invention relates to a computer implemented method and apparatus for cognitive testing, specifically for dementia-related afflictions such as Alzheimer's disease.

### BACKGROUND

Dementia is a progressive decline in cognitive function due to damage or disease in the brain over and above declines normally occurring due to aging. Dementia is more common in the elderly but can occur in any adult. Alzheimer's disease is the most common cause for dementia. Typically, areas of function affected by dementia include visuospatial skills, executive function, memory, attention, orientation, and verbal fluency. Therefore, it is essential to include these six cognitive domains in any cognitive screening test for dementia. In the case of Alzheimer's disease, each specific cognitive domain can be affected in a selective fashion. In approximately 50% of cases of Alzheimer's disease memory and verbal fluency are affected while the other cognitive domains remain substantially unaffected. Identifying such a pattern of cognitive abnormality, referred to herein as a “domain specific cognitive pattern,” is helpful in diagnosing various levels and causes of dementia.

Another type of dementia, vascular dementia, has a different domain specific cognitive pattern, which helps doctors differentiate between, for example, vascular dementia and Alzheimer's disease. Differentiating among the causes of dementia can be critical when attempting to diagnose the earliest form of Alzheimer's disease or mild cognitive impairment. Early identification of Alzheimer's disease usually results in early treatment and potentially delaying progression of cognitive impairment.

Of the early warning dementia tests, the Mini-Mental State Examination (MMSE), a paper and pencil test established in 1970, is well respected for screening and diagnosing cognitive impairment in patients. Specifically, it is the industry standard test for Alzheimer's diagnosis, but is also useful for testing impairment to cognitive thinking and other forms of dementia. The MMSE and most of the cognitive impairment tests available to health care professionals today do not, however, test all the cognitive domains discussed above, and therefore cannot provide a full picture of the patient's cognitive function. Specifically, some tests do not provide adequate testing to indicate early Alzheimer's disease and/or mild cognitive impairment. Furthermore, some tests available today do not take into account highly educated people who suffer only from mild dementia because such individuals may find the questions of the tests simplistic. Similarly, poorly educated people may indicate dementia when it is not truly present.

Additionally, most of the tests available today require a trained professional test administrator, which increases the cost of testing and adds constraints onto the times and locations available for testing. Yet other tests rely on olfactory testing because olfactory loss is an early indicator of Alzheimer's disease. Unfortunately, olfactory loss is caused by many conditions other than Alzheimer's disease, and is therefore an ineffective method of indicating Alzheimer's disease. Finally, many of the tests available today require a significant amount of time, some over one hour, to complete and therefore cost more to administer.

What is needed, therefore, is a method for testing a patient's cognitive function and a cognitive testing computer system that overcomes problems such as those described above, at least in part.

### SUMMARY

The above and other needs are met by a computer implemented method for testing a patient's cognitive function including visuospatial skills and executive function. The method includes displaying a message regarding the proper shape of a clock face, receiving input from the patient indicating a choice in response to the message regarding the proper shape of the clock face, displaying a message regarding the proper positioning of clock hands to indicate a specific time of day, receiving input from the patient indicating a selection of position of clock hands in response to the message regarding the proper positioning of clock hands, and evaluating the patient's visuospatial skills and executive function based at least on the input received.

In one embodiment, the method includes displaying a plurality of shapes of a clock face, at least one of which is a circle, before receiving input from the patient regarding the proper shape of the clock face. In other embodiments, the method also includes displaying a plurality of numbers including at least the numbers three, six, nine and twelve, displaying a message regarding the proper placement of the plurality of numbers on the clock face, and receiving input from the patient indicating a placement of the plurality of numbers in response to the message regarding proper placement of the numbers. In yet other embodiments, the method includes displaying a plurality of positions of clock hands before receiving input from the patient indicating a selection of position of clock hands.

In some embodiments, the method includes executing a software algorithm for generating workstation instructions on a computer server which is remote from a computer workstation, receiving the workstation instructions at the computer workstation, and executing the workstation instructions on the computer workstation to perform the method steps discussed above. In some embodiments, the displays of the method steps discussed above are performed using a web browser interface, and in others, the method steps are performed by a computer workstation with no interaction from a human test administrator.

The above and other needs are further met by a computer implemented method for cognitive testing of a patient comprising administering a clock drawing test. This method includes displaying a message regarding the proper shape of a clock face, receiving input from the patient indicating a choice in response to the message regarding the proper shape of the clock face, displaying a message regarding the proper positioning of clock hands to indicate a specific time of day, receiving input from the patient indicating a selection of position of clock hands in response to the message regarding the proper positioning of clock hands, and evaluating the patient's visuospatial skills and executive function based on the input received. The computer implemented method also includes administering a memory test including evaluating the patient's memory function, administering a verbal fluency test including evaluating the patient's verbal fluency, administering an attention test including evaluating the patient's attention function, administering an orientation test including evaluating the patient's orientation function, and compiling the evaluations performed in one or more of these evaluations into an overall test score indicating cognitive function of the patient.

In some embodiments, the method includes executing a software algorithm for generating workstation instructions on a computer server that is remote from a computer workstation, receiving the workstation instructions at the computer workstation, and executing the workstation instructions on the computer workstation to perform the previously described method steps. In yet other embodiments, at least one of the method steps is performed using a web browser interface, and in others, the method is performed by a computer workstation with no interaction from a human test administrator.

In some embodiments, administering the memory test also includes displaying a first message requesting the patient to memorize a memory item, discontinuing the display of the first message, displaying a second message requesting the patient to recall the memory item, receiving input from the patient indicating the patient's recall of the memory item, and evaluating the patient's memory function based at least in part on the input received. In other embodiments, administering the verbal fluency test also includes displaying a message requesting the patient to input a plurality of species names of a well-known genus, receiving input from the patient indicating the patient's response to the message, and evaluating the patient's verbal fluency based at least in part on the input received. In yet other embodiments, administering the attention test also includes displaying a plurality of characters, displaying a message regarding the characters, receiving input from the patient indicating the patient's response to the message, and evaluating the patient's attention function based at least in part on the input received. In yet other embodiments, administering the orientation test also includes displaying at least one message requesting the patient to input at least one item selected from the group consisting of the current year, the current month, and the current day of the week, receiving input from the patient in response to the at least one message, and evaluating the patient's orientation function based at least in part on the input received.

In one embodiment of the computer implemented method, administering the clock drawing test also includes indicating a choice regarding the proper shape of the clock face prior to receiving input from the patient, displaying a plurality of shapes at least one of which is a circle, displaying a plurality of numbers including at least the numbers three, six, nine and twelve, displaying a message regarding the proper placement of the plurality of numbers on the clock face, receiving input from the patient indicating a placement of the plurality of numbers in response to the message regarding proper placement of the numbers, and displaying a plurality of positions of clock hands prior to receiving input from the patient indicating a selection in response to the message regarding the proper positioning of clock hands.

In some embodiments, the overall test score indicates a level of cognitive impairment when the overall test score falls into a severe cognitive impairment range, a medium cognitive impairment range, a mild cognitive impairment range, or an early cognitive impairment range.

In some embodiments, the method includes displaying a welcome page before administering the clock drawing test including displaying at least one message regarding the patient and receiving input from the patient indicating a response to the message, and in others, the method includes storing the overall test score for analysis based on comparison with other patients' overall test scores. In yet others, the well-known genus includes animals and the plurality of species names includes everyday names of animals, and in others, the memory item is a word.

The above and other needs are further met by a cognitive testing computer system comprising means for administering a clock drawing test including means for displaying a message regarding the proper shape of a clock face, means for receiving input from the patient indicating a choice in response to the message regarding the proper shape of the clock face, means for displaying a message regarding the proper positioning of clock hands to indicate a specific time of day, means for receiving input from the patient indicating a selection of position of clock hands in response to the message regarding the proper positioning of clock hands, and means for evaluating the patient's visuospatial skills and executive function based on the input received. The system also includes means for administering a memory test including evaluating the patient's memory function, means for administering a verbal fluency test including evaluating the patient's verbal fluency, means for administering an attention test including evaluating the patient's attention function, means for administering an orientation test including evaluating the patient's orientation function, and means for compiling the evaluations performed in one or more of the steps described above into an overall test score indicating cognitive function of the patient.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a diagram of a cognitive testing network according to one embodiment of the invention.

FIG. 2 is a diagram of a computer workstation connected to a communication network according to one embodiment of the invention.

FIG. 3 is a flowchart of a computer implemented method for cognitive testing of a patient according to one embodiment of the invention.

FIG. 4A is a screenshot of a welcome page of the computer implemented method for cognitive testing of a patient according to one embodiment of the invention.

FIG. 4B is a flowchart of a welcome page method according to one embodiment of the invention.

FIGS. 5A and 5B are screenshots of first and second pages, respectively, of a clock drawing test of the computer implemented method for cognitive testing of a patient according to one embodiment of the invention.

FIG. 5C is a flowchart of a clock drawing test method according to one embodiment of the invention.

FIGS. 6A and 6B are screenshots of first and second pages, respectively, of a memory test of the computer implemented method for cognitive testing of a patient according to one embodiment of the invention.

FIG. 6C is a flowchart of a memory test method according to one embodiment of the invention.

FIG. 7A is screenshot of a verbal fluency test of the computer implemented method for cognitive testing of a patient according to one embodiment of the invention.

FIG. 7B is a flowchart of a verbal fluency test method according to one embodiment of the invention.

FIG. 8A is a screenshot of an attention test of the computer implemented method for cognitive testing of a patient according to one embodiment of the invention.

FIG. 8B is a flowchart of an attention test method according to one embodiment of the invention.

FIG. 9A is a screenshot of an orientation test according to one embodiment of the invention.

FIG. 9B is a flowchart of an orientation test method according to one embodiment of the invention.

FIG. 10A is a screenshot of a summary page according to one embodiment of the invention.

FIG. 10B is a flowchart of a summary page method according to one embodiment of the invention.

### DETAILED DESCRIPTION

A computer implemented method provides cognitive testing, specifically for dementia-related afflictions, such as Alzheimer's disease. This computerized self test, which is administered on a computer workstation in an interactive, web-based format, screens the cognitive abilities of the test-taker or “patient.” The test includes evaluation of six areas of cognitive domain including visuospatial skills, executive function, memory, verbal fluency, attention, and orientation, all of which are essential for indications of dementia, including Alzheimer's disease and other cognitive afflictions. The test does not require administration by a trained professional, but rather, is easily self-administered assuming the patient has rudimentary computer skills. The test is administered in a web browser, either from a server to the workstation via the Internet or locally at the workstation, and is configured to be completed within ten minutes. If necessary, a caregiver, family member or primary care physician in a home or office setting can administer the test.

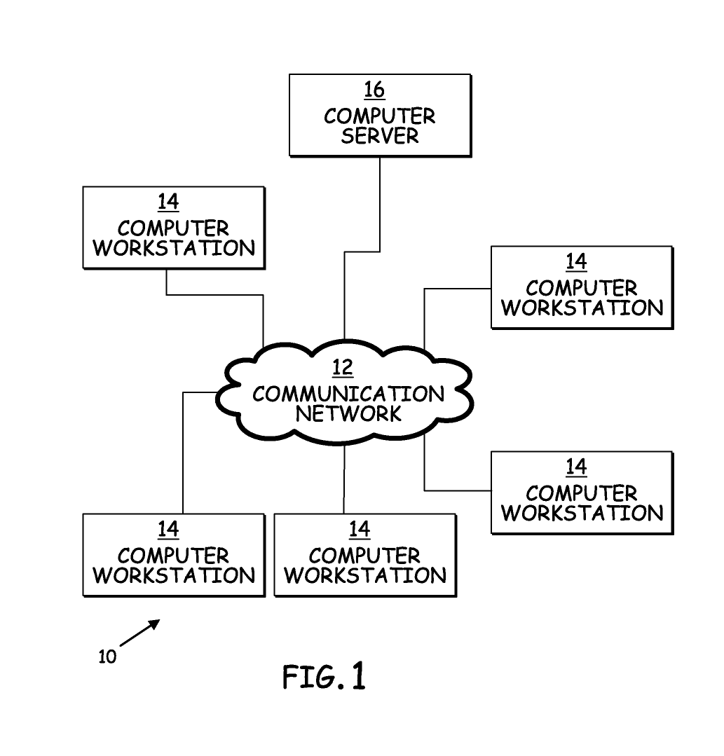

Referring now to FIG. 1, a cognitive testing network 10 is shown. The cognitive testing network 10 includes a communication network 12 such as the Internet and a plurality of computer workstations 14 connected to the communication network 12. Typically, a server 16 is connected to the communication network 12 also. In some embodiments, the server 16 processes a software algorithm to implement the method for cognitive testing described in further detail below, and the computer workstations 14 receive the instructions from the software algorithm over the communication network 12. In other embodiments, an individual computer workstation 14 runs the software algorithm to implement the method for cognitive testing locally. That is, the software algorithm is stored locally at the computer workstation, processed locally, and administered locally.

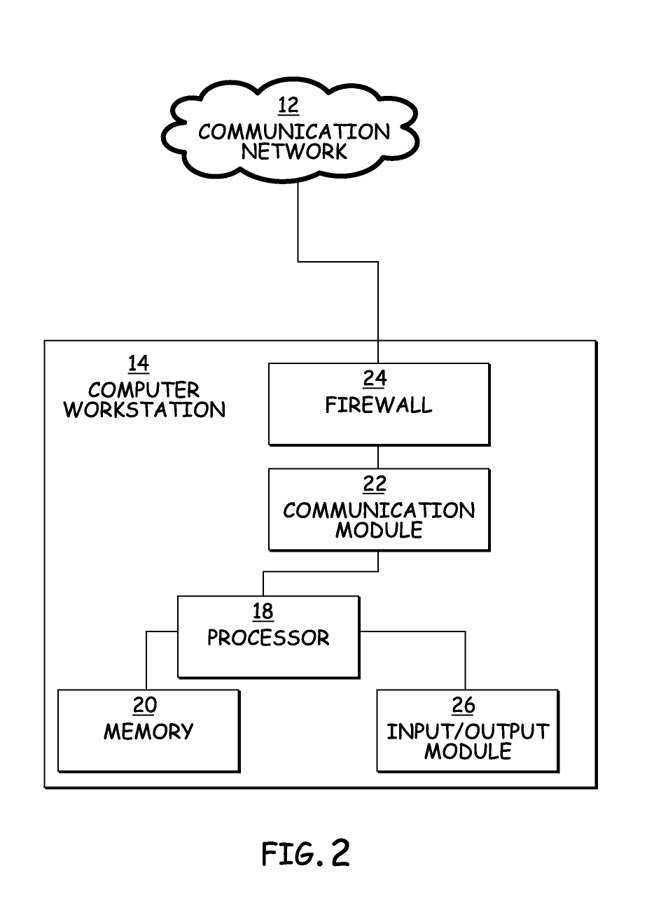

Referring now to FIG. 2, the computer workstation 14 is shown in expanded view. The computer workstation, in some embodiments, has a processor 18 connected to a memory 20. The processor is also connected to a communication module 22, which communicates with the communication network 12 through a firewall 24 in some embodiments. Also, the processor 18 is connected to an input/output module 26 for interacting with a user such as the patient.

The processor 18 processes a software algorithm as further discussed below. Based on the instructions from the software algorithm, the processor 18 controls the communication module 22 and the input/output module 26 to function in the cognitive testing network 10 or as a stand-alone cognitive testing workstation. The input/output module 26 typically includes a visual display such as a monitor, a keyboard, and a mouse.

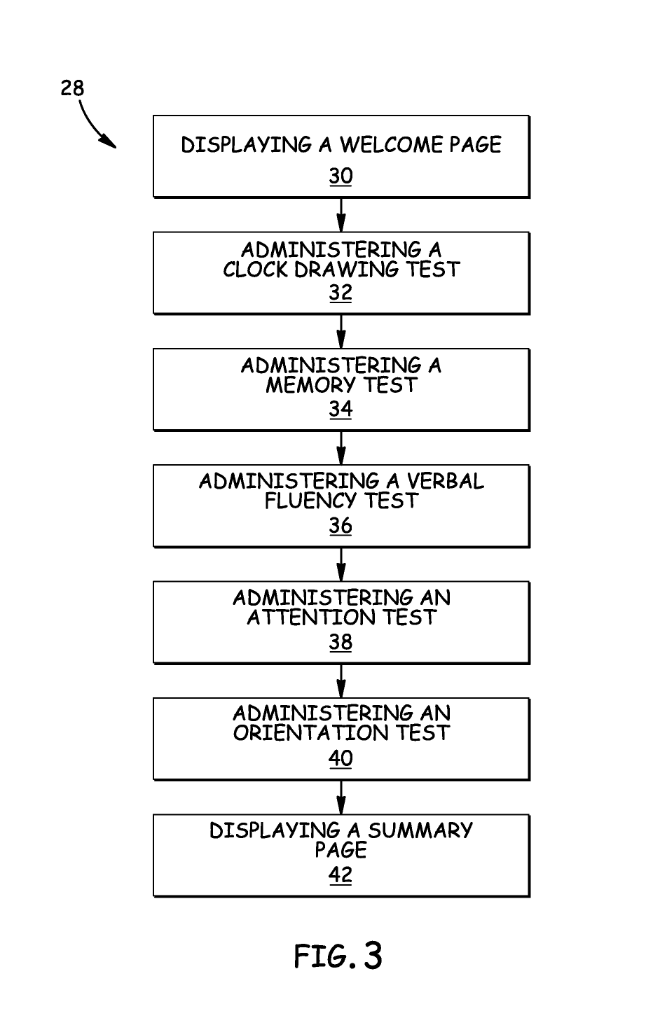

Referring now to FIG. 3, a flowchart illustrating one embodiment of a computer implemented method 28 for cognitive testing of a patient is shown. First, a welcome page is displayed (step 30). Next, various domains for screening dementia are tested in succession, beginning with administering a clock drawing test for evaluating visuospatial skills and executive function (step 32). Then a memory test is administered (step 34), a verbal fluency test is administered (step 36), an attention test is administered (step 38), and an orientation test is administered (step 40). Finally, an overall test score is compiled indicating cognitive function of the patient, and the overall test score is displayed on a summary page (step 42).

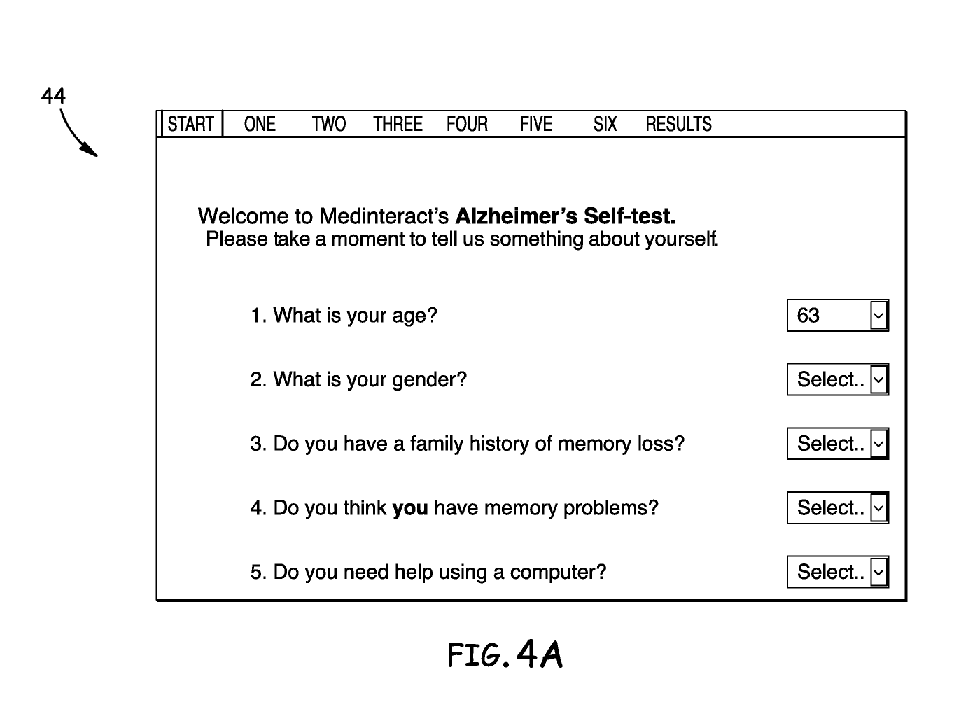

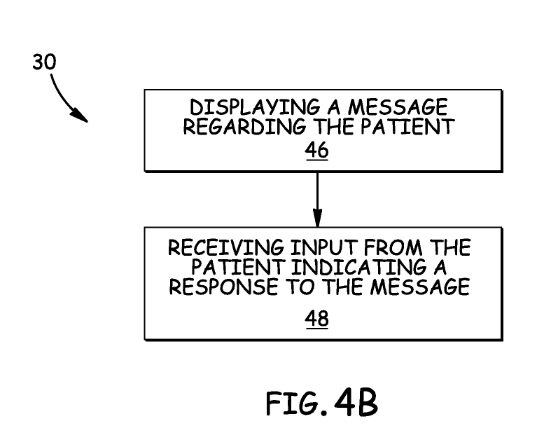

Referring now to FIGS. 4A and 4B, the welcome page 44 is described. FIG. 4A shows a screenshot of one embodiment of the welcome page 44, and FIG. 4B illustrates one embodiment of the welcome page method 30. The welcome page typically includes general questions which illicit responses from the patient that are stored and later used for comparative analysis as discussed further below. Referring to FIG. 4B, at least one message regarding the patient is displayed (step 46). Second, input from the patient is received indicating a response to the message (step 48). In most embodiments, demographic information is requested in step 46 such as the patient's age, gender, etc. Furthermore, inquiries into the patient's family history regarding memory loss and the patient's opinion of his or her personal memory ability are included in some embodiments. Responses received in step 48 to the personal and demographic questions are stored at the computer workstation 14 for subsequent comparative analysis. For example, the test scores of patients within a similar demographic group as determined by the patients' responses to the demographic questions may be compared. Also, one or more questions are displayed in some embodiments regarding the patient's ability to self-administer the test. In response to the questions, the patient has the opportunity to request human assistance in taking the test. However, if the patient responds that no help is needed, the test is administered with no interaction from a human test administrator.

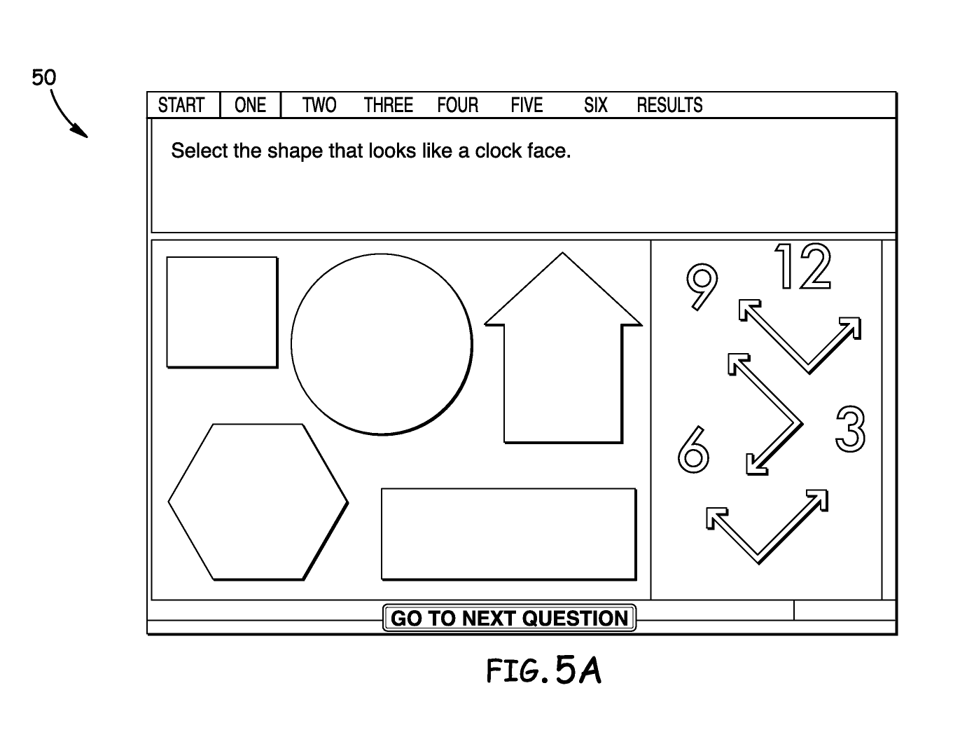

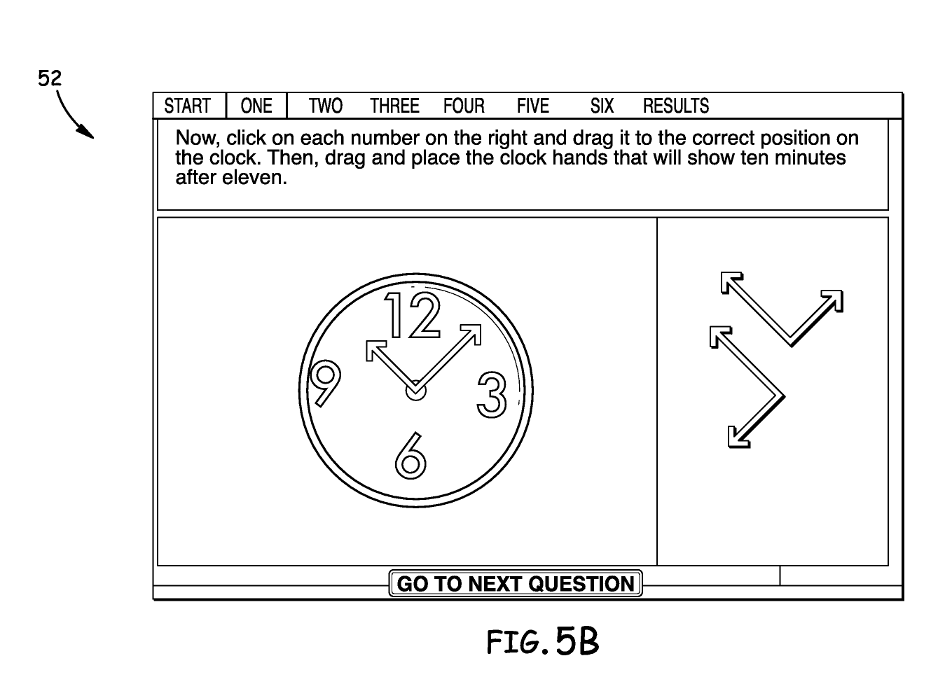

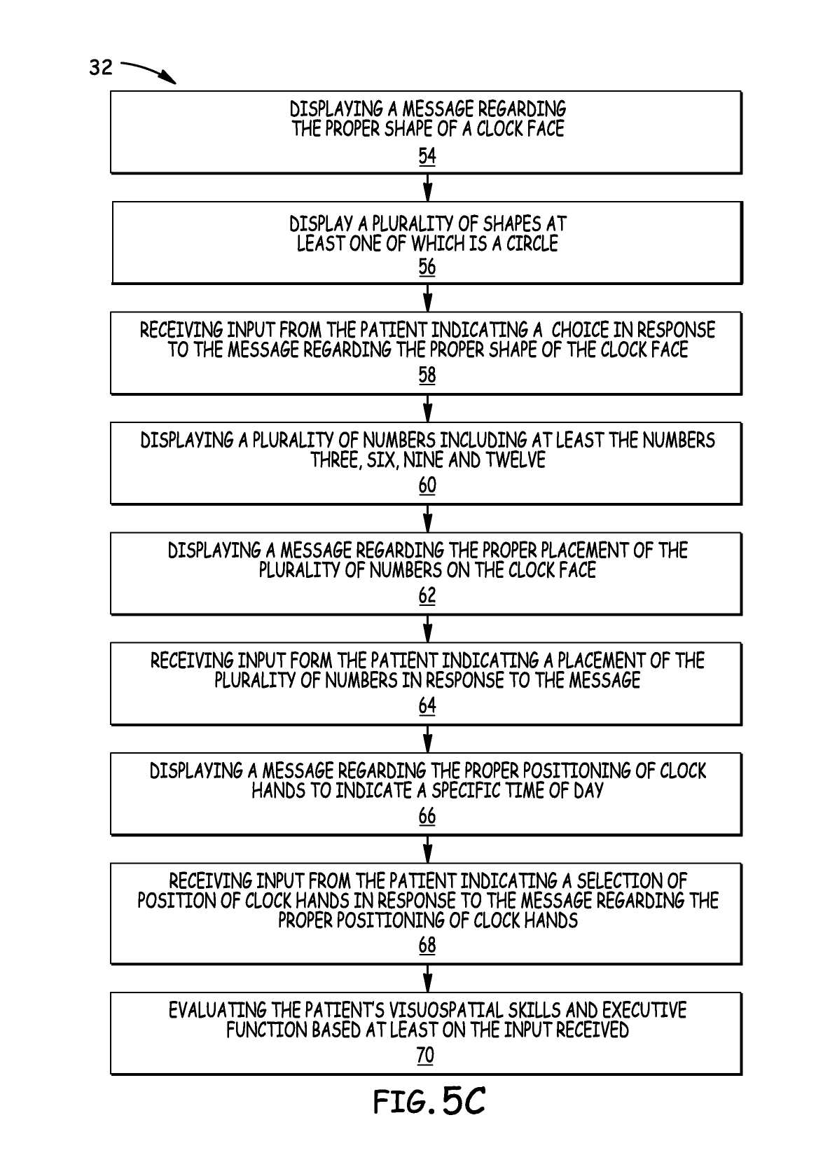

Referring now to FIGS. 5A, 5B and 5C, the clock drawing test assesses a patient's visuospatial skills and executive function. FIG. 5A is a screenshot of the first page of the clock drawing test 50, FIG. 5B is a screenshot of the second page of the clock drawing test 52, and FIG. 5C illustrates one embodiment of the clock drawing test method 32.

First, a message regarding the proper shape of a clock face is displayed (step 54). One embodiment of the message, as shown in FIG. 5A, requests the patient to select the shape that most resembles a clock face. Next, a plurality of shapes, at least one of which is a circle, are displayed (step 56). In one embodiment, the message of step 54 and the shapes of step 56 are displayed substantially simultaneously. The patient then indicates a choice in response to the message regarding the proper shape of the clock face by providing input, which is received by the computer workstation (step 58). The input is entered by the patient by moving a mouse and clicking on the chosen shape in one embodiment. In another embodiment, the shapes are displayed on a touch-screen capable of receiving tactile input from the patient. In such an embodiment, the patient indicates his or her choice by pressing a finger or other physical object onto the portion of the touch-screen displaying the chosen shape. In yet other embodiments, the patient enters his or her choice using a keyboard.

Once the workstation has received the patient input, the incorrect clock face shapes are removed from the display and the proper clock shape face, that is, the circle remains and is enlarged as shown in FIG. 5B. Also, a plurality of numbers are displayed (step 60). In the embodiment shown in FIG. 5A, the plurality of numbers are displayed concurrently with the plurality of shapes, but in other embodiments, the plurality of numbers appear concurrently with the removal of the incorrect clock face shapes and/or subsequent to the removal of the incorrect clock face shapes. In the particular embodiment shown in FIG. 5, the numbers three, six, nine, and twelve are displayed, but in other embodiments, all the numbers one through twelve or different variations of clock face numbers are displayed. In other embodiments, as discussed below, the numbers are displayed individually, that is, separately from one another, such that the patient makes a choice regarding the position of one number before the next number is displayed.

Next, a message regarding the proper placement of the plurality of numbers on the clock face is displayed (step 62) as shown in FIG. 5B. The message in this embodiment asks the patient to click and drag each number on the right to the correct position on the clock. Then, the patient indicates a placement of the plurality of numbers in response to the message by providing input to the workstation. The workstation receives the input (step 64) via mouse, touch-screen, keyboard or other means. In embodiments using a touch-screen, the patient, with his or her finger, touches the area of the screen displaying a number and slides his or her finger to the chosen position on the clock face for the number.

Next, a message regarding the proper positioning of clock hands to indicate a specific time of day is displayed (step 66). The message in the embodiment of FIG. 5B is a request for the patient to drag and place the clock hands to a position showing ten minutes after eleven. The patient indicates a selection of position of clock hands in response to the message by dragging the chosen clock hands to the chosen position on the clock face, and the workstation receives the placement as input (step 68). Finally, the workstation evaluates the patient's visuospatial skills and executive function based on the input received (step 70).

Regarding step 58, the patient's response is graded based on whether the correct shape was chosen. If the patient chose the circular shape, one point is awarded. Regarding steps 64 and 68, the numbers and clock hands are dragged and dropped into the positions chosen by the patient. Such positions are recorded as a relative (x,y) coordinate system. The dropped positions of the items are graded based on where they are dropped relative to the center of the circular clock face. Specifically, in one embodiment, the circular clock face is divided into quadrants each centered about 0, 90, 180, and 270 degrees, respectively, as measured from a horizontal axis (through the center of the clock face and also through the proper positions of both the numbers 3 and 9 on the clock face) in the counter-clockwise direction. The recorded (x,y) position of each number is tested to determine whether it falls into the proper quadrant. For example, the number 12 should fall within the quadrant centered about 90 degrees, that is, it should fall between 45 and 135 degrees. Likewise, the number 9 should fall in the quadrant centered about 180 degrees or between 135 and 225 degrees. The number 6 should fall in the quadrant centered about 270 degrees or between 225 and 315 degrees. Finally, the number 3 should fall in the quadrant centered about 0 degrees between 315 and 45 degrees. In one embodiment, if the patient successfully drags each of the four numbers into the proper respective quadrants of the clock face, the patient is awarded one point.

Furthermore, the location is tested to ensure the number has been placed within a reasonable distance from the center of the circle clock face. For example, in one embodiment, the numbers must be placed within 125% of the diameter of the clock face, allowing the numbers to be placed a short distance outside the perimeter of the circular clock face and still indicate a successful placement. In one embodiment, if the patient successfully drags each of the four numbers reasonably close to the center of the clock face, the patient is awarded one point.

Regarding step 68, if the patient chooses the correct clock hands to indicate the time requested and the axis of the hands reasonably corresponds to the center of the clock face, the patient is awarded one point. In one embodiment, the axis of the clock hands can be dropped within a distance equivalent to 37.8% of the diameter of the clock face in order to reasonably correspond to the center of the clock face. Thus, the maximum score, in the embodiment described above, for the clock drawing test is four points.

In other embodiments of the clock drawing method 32, multiple clock hands are displayed alongside the clock face and numbers in some embodiments and without display of the clock face and numbers in other embodiments. A message is displayed requesting the patient to choose the clock hands that indicate a specific time of day, for example ten after eleven. Once the patient has made a choice regarding the clock hands, without dragging the clock hands onto the clock face, the test moves on to the next page. In other words, the patient does not have the opportunity to click and drag each of the clock hand choices onto the clock face in order to make a choice. Rather, the patient must make a choice from the clock hands on the display without dragging and associating the clock hands with the clock face on the display.

In other embodiments of the clock drawing method 32, the plurality of numbers displayed in step 60 are displayed one at a time beginning with 3 and continuing through 12. In some of these embodiments, the numbers appear out of numerical order, for example, the order (6, 3, 12, 9). The message of step 62 requests the patient to click on the clock face where the number should be. Once the user clicks on the clock face (step 64), the number automatically moves to the clicked location, and the scoring is handled as described above. Similarly, in some embodiments, the user is asked to select the proper clock hands (step 66) to indicate a specific time of day. Then the user is asked to click on the proper location for the clock hands to be placed on the clock face (also step 66). Again, scoring is handled as described above. These embodiments obviate the necessity of the patient “dragging” the numbers and the clock hands into their proper positions, which may be a beneficial characteristic of the method in cases where the patient has minimal computer skills.

In another embodiment, the scoring is handled differently than described above. In this embodiment, if the patient positions at least one of the numbers in the proper quadrant and a reasonable distance from the center of the clock face, the patient is awarded one point. If the patient positions each of the four numbers in their proper respective quadrants all at reasonable distances from the center of the clock face, the patient is awarded two points.

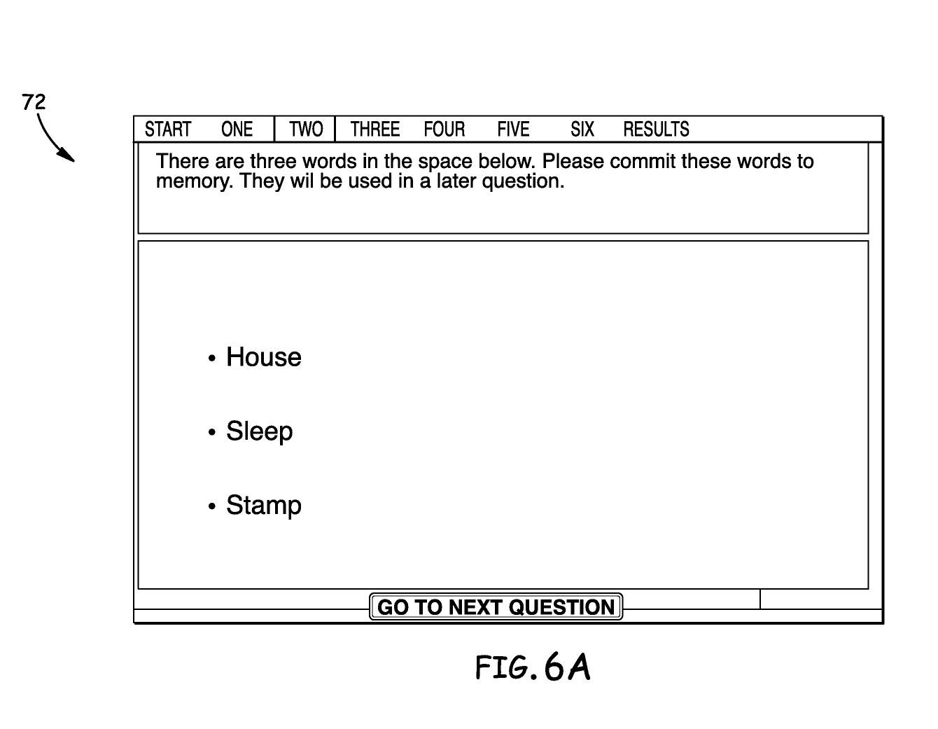

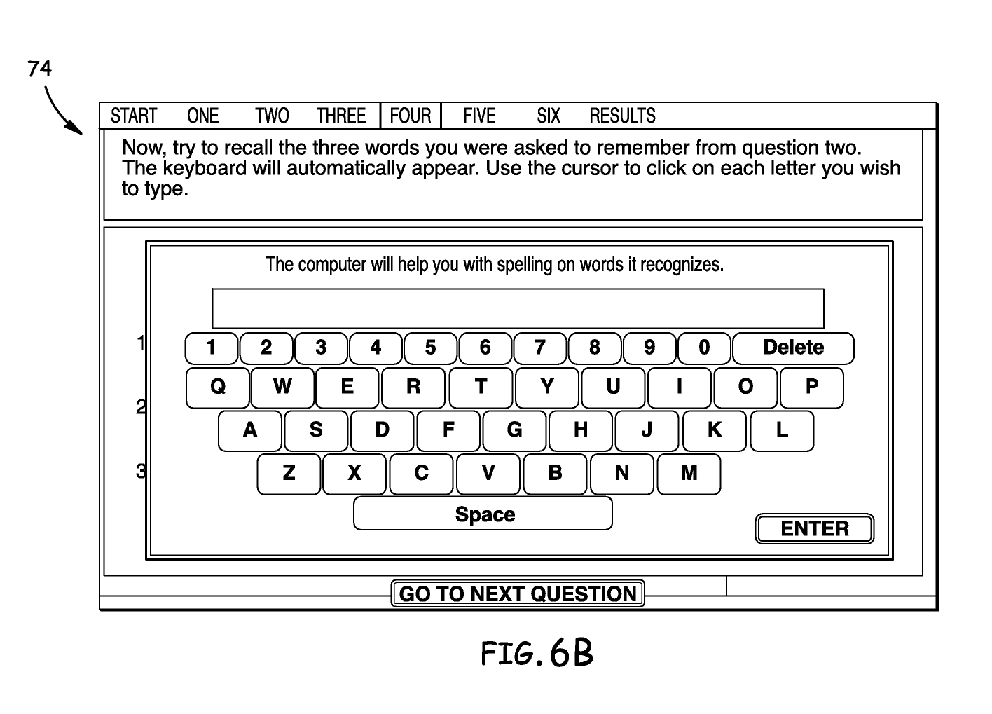

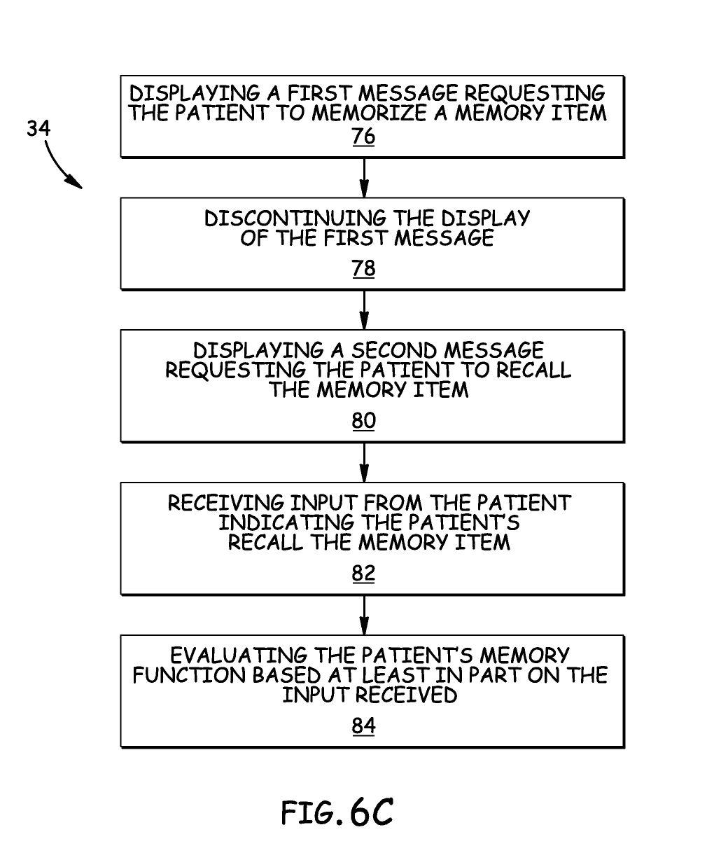

Referring now to FIGS. 6A, 6B and 6C, the memory test assesses a patient's memory function. FIG. 6A is a screenshot of the first page of the memory test 72. FIG. 6B is a screenshot of the second page of the memory test 74, and FIG. 6C illustrates one embodiment of the memory test method 34. First, a message requesting the patient to memorize a memory item is displayed (step 76). The message in the embodiment shown in FIG. 6 requests the patient to memorize three words and indicates that the words will be used in a later question.

In one embodiment, the three words are randomly chosen from a database including fifteen words so that the three words to be memorized will vary in subsequent administrations of the test. In other embodiments, different numbers of memory items are memorized and the memory items vary from words to icons of animals, foods, or other well-known and easily recognizable items.

Next, the first message display is discontinued (step 78) so that the patient must remember the memory item without reference to the first message. In some embodiments, intervening messages and/or tests are performed, such as the verbal fluency test described below. Then a second message requesting the patient to recall the memory item is displayed (step 80). One embodiment of step 80 is shown in FIG. 6B. In this embodiment, the second message asks the patient to recall the three words previously displayed in step 76. Next, input is received from the patient indicating the patient's recall of the memory item (step 82), which in this embodiment consists of three words. A virtual keyboard is displayed in some embodiments in order that the patient may click on letters to spell his or her selection. Finally, the patient's memory function is evaluated based at least in part on the input received in the previous steps (step 84).

The patient is evaluated (step 84) based on unique matches input by the patient to the memory items displayed in step 76. Thus, in the embodiment described including three words, the patient is awarded one point for each of the unique words the patient correctly inputs in step 82. In other words, if the patient inputs a correct response of “house” three times, the patient will only be awarded one point. However, if the patient inputs three correct responses including “house,” “sleep,” and “stamp,” the patient will be awarded three points.

In some embodiments, a time limit, such as 30 seconds, is imposed on the display of step 76, and step 78 automatically occurs without the patient indicating his or her desire to proceed. Thus, the patient is given a limited amount of time to commit the memory item to memory. Also, in some embodiments, the display of step 80, the input received in step 82 and the evaluation of step 84 are performed after other portions of the cognitive testing method 28. For example, the verbal fluency method 36 described below is performed between discontinuing the display of the first message (step 78) and displaying a second message requesting the patient to recall the memory item (step 80). This provides a period of time in which the patient must focus on another cognitive domain test and then revisit the memory items requested to be memorized, which typically better indicates the patient's memory function than quickly requesting a patient to recall words recently memorized, for example in a previous step of the method.

Notably, in some embodiments, the words used in the memory test method 34 are saved for subsequent analysis if necessary. For example, a physician reviewing the test results may be interested in reviewing the words used in the memory test in performing his or her diagnosis.

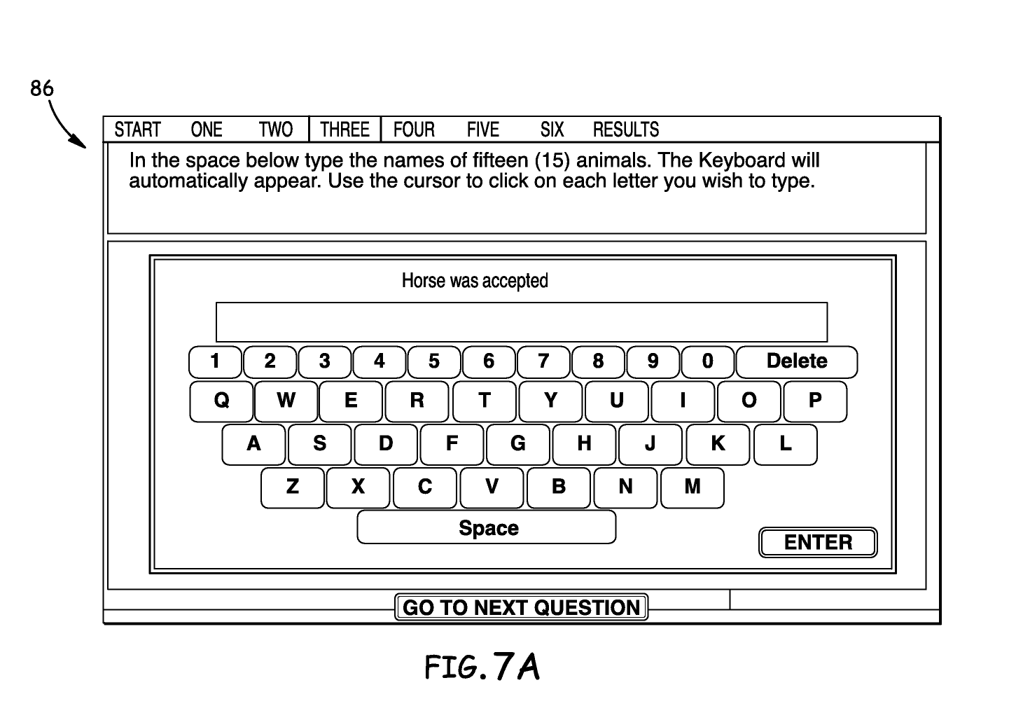

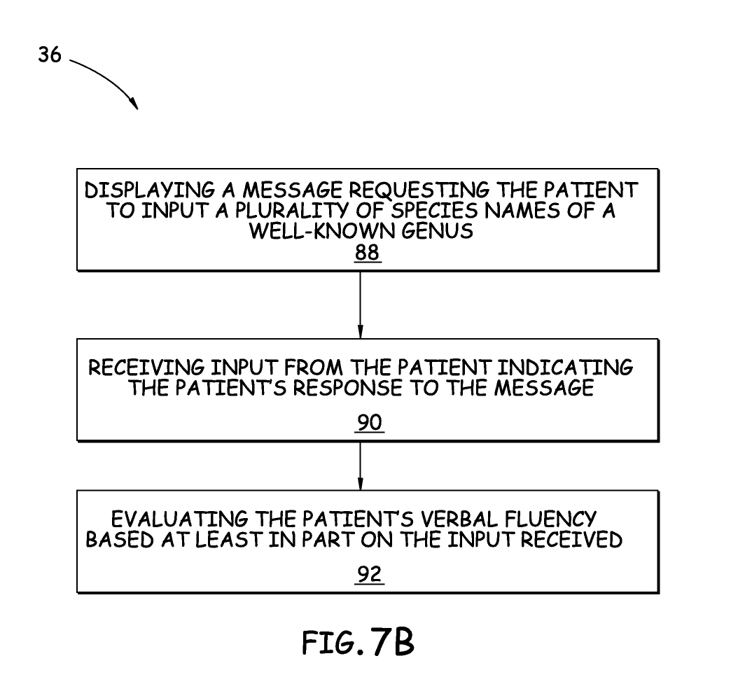

Referring now to FIGS. 7A and 7B, the verbal fluency test assesses a patient's verbal fluency. FIG. 7A is a screenshot of the verbal fluency test 86, and FIG. 7B illustrates one embodiment of the verbal fluency method 36. The first step 88 is displaying a message requesting the patient to input a plurality of species names of a well-known genus. In the embodiment shown in FIG. 7A, the well-known genus is animals and the species names, of course, are names of animals. A list of animals is stored in a database associated with the method algorithm. The patient indicates the patient's response to the message and the workstation receives the input (step 90). In the embodiment shown in FIG. 7A, a virtual keyboard is displayed allowing the patient to click on letters to spell his or her selection. Finally, the patient's verbal fluency is evaluated based at least in part on the input received (step 92).

In one embodiment of the verbal fluency method 36, the patient is briefly shown a list of fifteen blank spaces and is asked to name fifteen animals (step 88). The virtual keyboard appears approximately three seconds later and the patient input is received (step 90). The patient's responses are matched to a database (also referred to herein as a “potential input database”) of over 1000 animals. In some embodiments, the method includes automatic completion of the animal name so that once the patient has input a portion of the animal name, the remainder is automatically completed. The method accepts complete animal names regardless of whether the complete animal name is a portion of a longer animal name. For example, “ant” is part of “antelope” and “anteater.” Once “ant” has been entered and accepted, the method allows “antelope” or “anteater” to be typed in their totality. That is, the method does not automatically accept “ant” as the patient's answer and thereby dock the patient a point for repetitive answering, but rather, allows the completion of the longer animal names. However, if an animal has been entered previously, and the patient attempts to type the same animal, the method typically will automatically complete the animal name. This saves time once the patient has demonstrated his or her input by partially spelling the name, particularly if the patient has trouble using the virtual or physical keyboard. In most embodiments, the patient is awarded one point for each correct, unique input for a total possible score of 15.

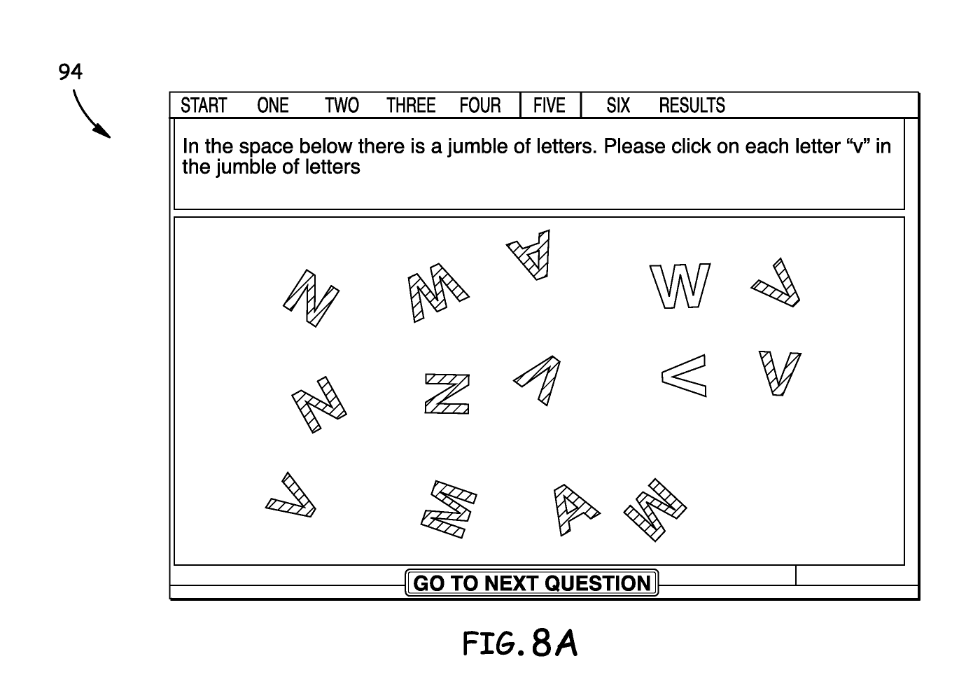

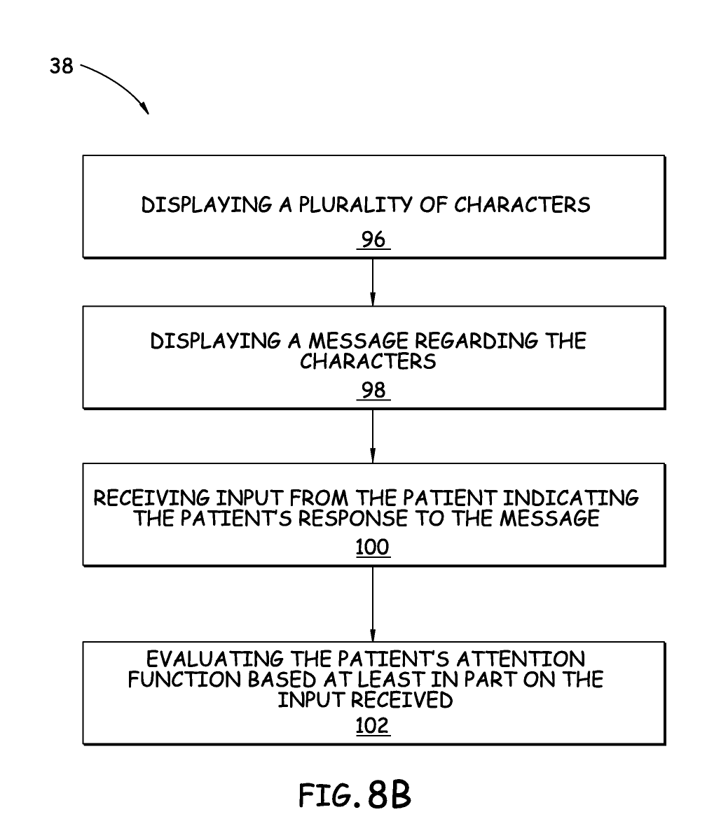

Referring now to FIGS. 8A and 8B, the attention test screens a patient's attention function. FIG. 8A is a screenshot of one embodiment of the attention test 94, and FIG. 8B illustrates one embodiment of the attention method 38. The first step 96 is displaying a plurality of characters. In the embodiment shown in FIG. 8A the characters are alpha-numeric characters and they are arranged in a plurality of orientations and positions. Also, the characters include a plurality of specific similar characters, which, in this embodiment, are “V”s. Next, a message regarding the characters is displayed in step 98. In the embodiment shown, the message asks the patient to “click on each letter ‘V’ in the jumble of letters.” Next, input is received from the patient indicating the patient's response to the message (step 100). In the embodiment shown, the patient chooses the “V”s from the jumble of letters. Finally, the patient's attention function is evaluated based at least in part on the input received in the previous steps (step 102).

In one embodiment of the attention test method 38, the screen is broken into an invisible grid having 14 squares. The system selects an alpha-numeric character at random from a database of images. However, the system always selects five of the characters the patient is requested to choose in steps 98 and 100. Also, the orientation of all the characters is chosen at random. When the patient selects a character, the character changes color to indicate the character has been chosen. If the patient chooses a previously chosen character, it returns to its original color to indicate that it is no longer chosen. Once five characters are selected, or after a predefined time limit, for example one minute, or after the patient chooses to go on to the next question, attention function is evaluated (step 102). The patient gets one point for each of the correct choices made. In the example, the patient receives one point for each “V” chosen up to five points.

In some embodiments, the system records the complete arrangement of characters and records the patient's choices and the times the choices were made. This information can be useful to a physician who is reviewing the test results in order to determine a diagnosis.

In another embodiment of the attention test, a message is displayed requesting the patient to input the names of two or more months starting with a particular month and moving backward in time. For example, the patient is asked to begin with December and name five months sequentially moving backward through the calendar. In this example, a fully correct response would be [December, November, October, September, August]. In one embodiment, all twelve months are displayed in a random and jumbled configuration. Also, five input boxes are displayed for input of the chosen months, and the first input box is highlighted to indicate it is ready to receive the patient's chosen month. When chosen, a month appears in the first input box and disappears from the jumble of months. Then, the highlight is removed from the first input box and the second input box is highlighted. Further, in some embodiments, the first input box, including the patient's first chosen month, is blacked-out in order that the patient cannot refer back to the patient's choice, but rather, must remember his or her first choice in order to make the second choice. This process is repeated until the patient has chosen five months. Upon each choice of the patient, the chosen month is removed from the jumble of months. The chosen month is displayed briefly in the corresponding input box and is then blacked-out.

In scoring this embodiment of the attention test, one point is awarded for each correct month for a total of five points. Alternatively, if the patient inputs an incorrect month, then continues to input months correctly from that point forward, the patient receives a point for each month that was entered correctly with regard to the previously entered month. For example, if the patient enters [December, August, July, June, November], one point is awarded for each of December, July, and June and no point is awarded for August or November.

Furthermore, in some alternatives of this embodiment of the attention test, a virtual keyboard, such as the virtual keyboard described above, is displayed and the patient is asked to input the months via the virtual keyboard or the physical keyboard.

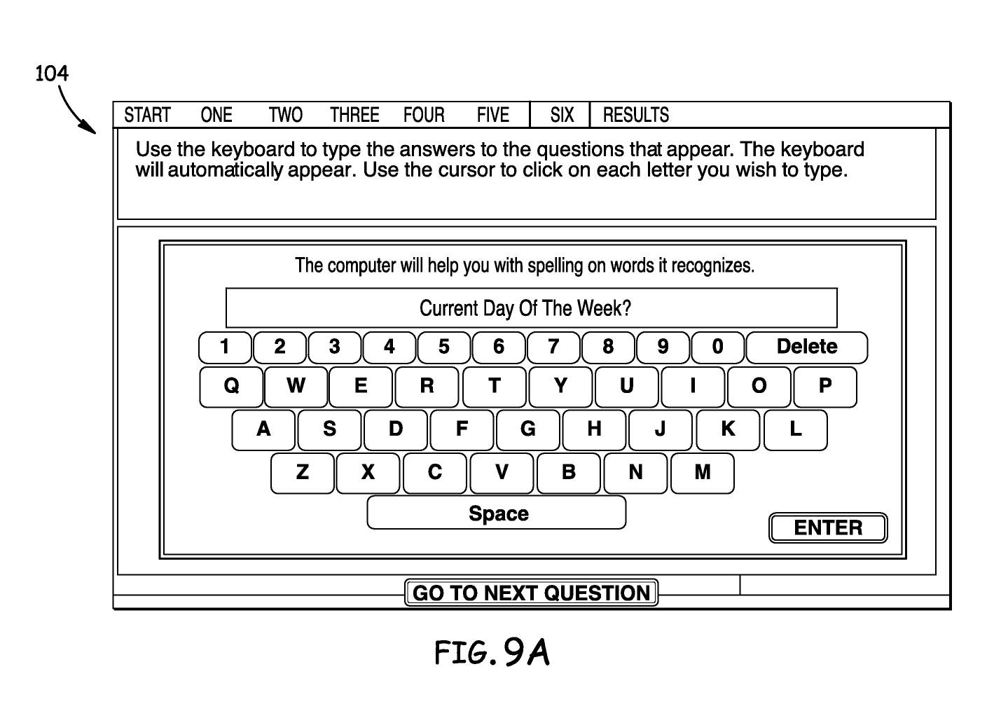

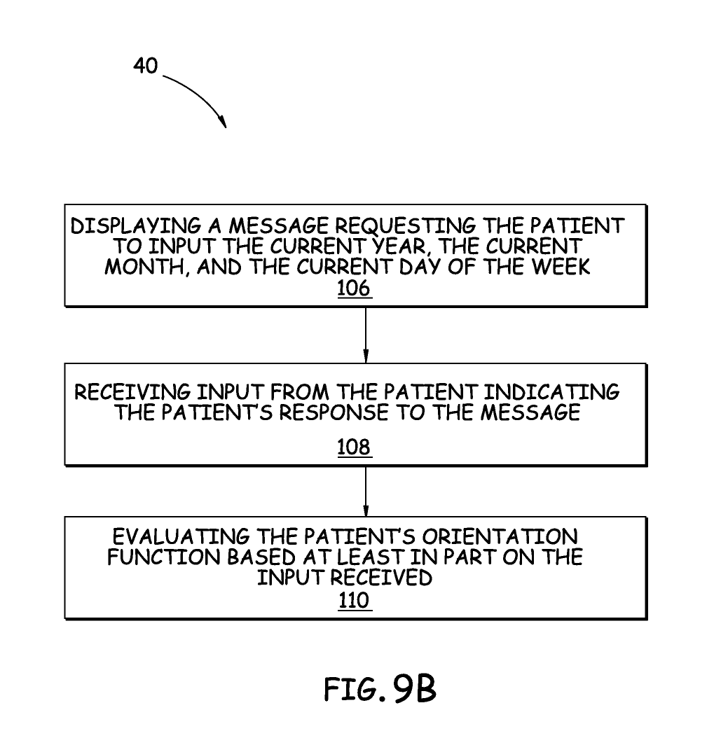

Referring now to FIGS. 9A and 9B, the orientation test assesses a patient's orientation function. FIG. 9A is a screenshot of one embodiment of the orientation test 104, and FIG. 9B illustrates one embodiment of the orientation test method 40. First, a message is displayed requesting the patient to input the current year, the current month, and the current day of the week (step 106). Next, input is received from the patient indicating the patient's response to the message (step 108). In the embodiment shown, a virtual keyboard is provided for the patient to click on letters in order to spell the patient's choice. Otherwise, the patient can enter his or her response via a physical keyboard. In some embodiments, step 106 includes multiple messages, where each requests the patient to input one piece of information. For example, a first message requests the current year, a second message requests the current month, and a third message requests the current day of the week. In such embodiments, step 108 is typically broken into multiple inputs, where each is received subsequent to the corresponding request but prior to the next request. Finally, the patient's orientation function is evaluated based at least in part on the input received in the previous steps (step 110).

In one embodiment of the orientation test method 40, the system attempts to match the patient's input in response to the request to input the current month to a list of months. Likewise, the system attempts to match the patient's input in response to the request to input the current day of the week to a list of days. Once a match has been made, the system automatically completes the entry and accepts it as input. The patient is evaluated based on correct responses to the three inquiries for a total possible score of three points.

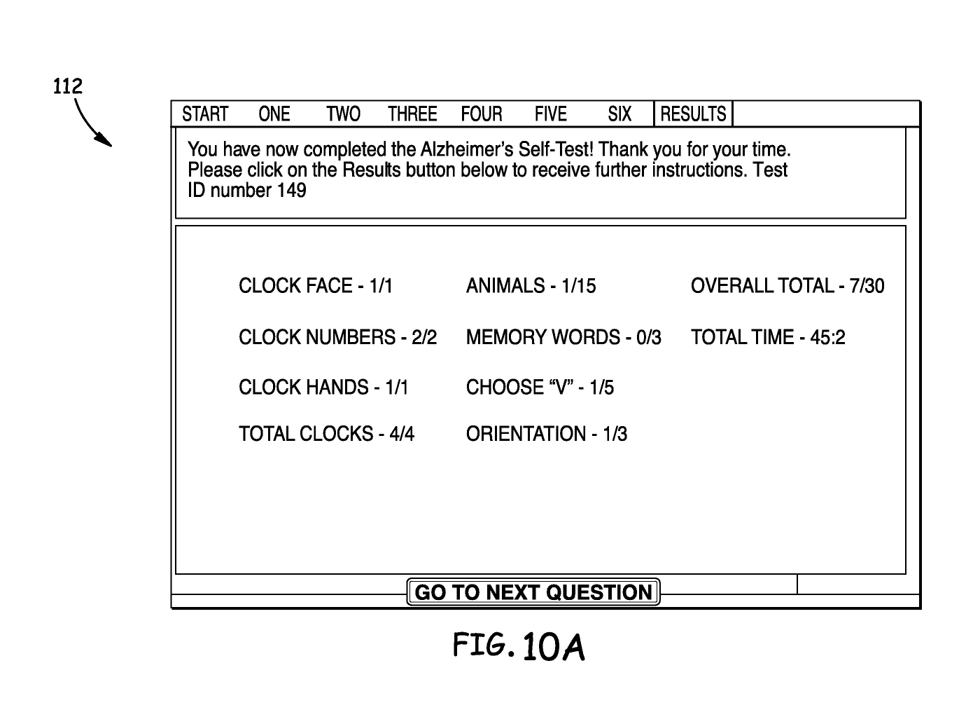

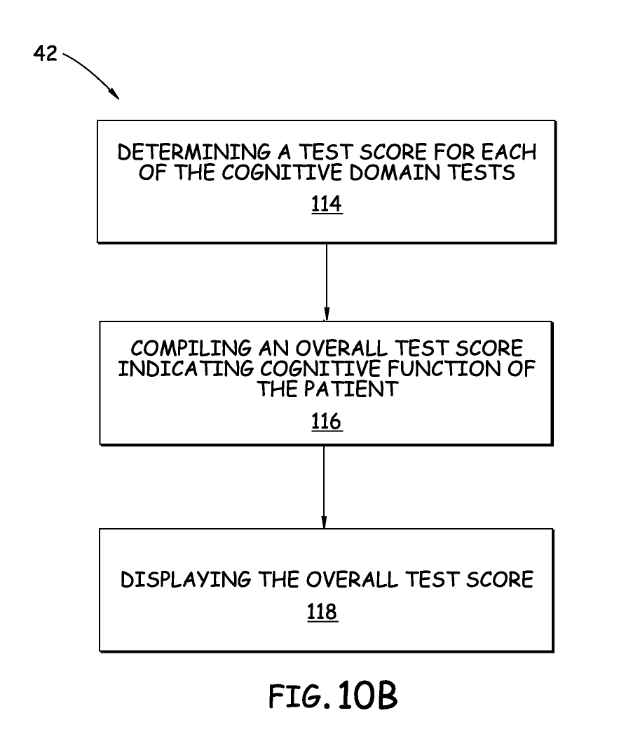

Referring now to FIGS. 10A and 10B, a summary page 112 displays a summary of the cognitive test. First, a test score for each of the cognitive domain tests is determined (step 114). In some embodiments, the determination for each individual cognitive test is included in the evaluation of the patient's function with regard to that specific test. In such cases, the determined individual test score is stored and retrieved in step 114. Next, the individual test scores for each of the cognitive domain tests are combined resulting in an overall test score (step 116). Finally, the overall test score is displayed (step 118). As shown in FIG. 10A, in some embodiments, the individual cognitive domain test scores are also displayed. Furthermore, in some embodiments, the total time elapsed during the taking of the cognitive test is displayed.

In some embodiments of the summary page method 42, the summary page does not display individual cognitive domain test results, but rather gives the patient a general message concerning their results and, in some embodiments, the patient is informed of the total test score. In some embodiments, the patient is given a message indicating the problem areas indicated by the test results, such as “it appears you might have a problem with verbal fluency.”

However, in some embodiments, the complete test is scored out of a possible 30 points and is correlated to with the Mini-Mental State Examination (MMSE) scoring, which is also out of 30 points. The scoring is configured to be comparable to MMSE scoring so that it provides a meaningful result to physicians and other health care professionals already familiar with the MMSE scoring standard. While the summary page typically does not display information indicating the possible levels of cognitive impairment to the individual, physicians will understand various ranges of scores to indicate that the patient is likely to be afflicted with particular levels of cognitive impairment. For example, severe cognitive impairment, such as severe Alzheimer's disease is indicated by an overall score of 10 or below. Moderate to severe cognitive impairment and/or moderate to severe Alzheimer's disease is indicated by an overall score of 11-20, mild to moderate cognitive impairment and/or mild to moderate Alzheimer's disease is indicated by an overall score of 20-25, and early cognitive impairment and/or early Alzheimer's disease may be indicated by an overall score of 26-30. As discussed above, however, the individual cognitive domain scores, which are typically stored for reporting to the diagnosing physician, are useful in diagnosing the various causes and types of dementia related afflictions.

In some embodiments of the test scoring, a normal or control group is included as well. For example, early cognitive impairment and/or early Alzheimer's disease is indicated by an overall score of 26-28 and normal cognitive function is indicated by a score of 29-30. In other embodiments of the scoring, various ranges are used to indicate the various levels of impairment.

The test results in some embodiments are automatically transmitted over the cognitive test network 10 to a physician's workstation so that the physician has access to the test results for purposes of assisting diagnosis. In other embodiments, the patient prints a hardcopy of the summary page and provides the summary page to the physician. In yet other embodiments, the test results are stored on the workstation computer and an authorized health care professional retrieves the test results as necessary.

In some embodiments of the summary page method 42, graphical tools such as bar graphs and line graphs are displayed. The graphical tools illustrate a correlation between the patient's test results against those of the patient's demographic group. For example, in one embodiment, the patient's results are compared to the average results of other patients in the same age group, for example, age 60 to age 70. Additionally, the comparison in other embodiments includes comparison between the patient's results and those of the same gender group. In yet other embodiments, multiple demographic characteristics of the patient are combined forming the patient's demographic profile. The patient's results are then compared to those other patients fitting the same demographic profile as the patient. Such graphical tools, in some embodiments, are stored at the workstation and in some embodiments are communicated to the patient's physician for further analysis and potential use in diagnosis.

In other embodiments, the domain specific scores discussed above are evaluated individually and combined to determine domain specific cognitive patterns resulting in different test results than the above method whereby the domain specific scores are added and a scoring range is determined. The detailed summary report discussed above is displayed in some applications and, in addition, one or more multimedia presentations are automatically communicated to the patient simultaneous or subsequent to viewing the summary page. The multimedia presentations are stored on the workstation typically and include audiovisual files such as MPEG, MOV, and the like. If a particular domain specific cognitive pattern is indicated by the test results, a multimedia presentation is chosen to correlate with the domain specific cognitive pattern indicated. In one embodiment, many multimedia presentations are stored at the workstation each correlating with a domain specific cognitive pattern and each is communicated via display and/or audio to the patient at or after the summary page.

The multimedia presentations are typically recordings of a physician specialist in the field of cognitive impairment discussing the test results with the patient. The discussion focuses on the one or more domain specific cognitive pattern(s) indicated by the test results, if applicable. If not applicable, general test results discussion, for example, the implications of indicating a mild to severe cognitive impairment is discussed. The content also typically includes suggestions for arranging a face to face meeting with a physician and instructions for the patient going forward.

In some embodiments of the method, when the patient is inputting responses to messages for the various cognitive domain tests, the responses are automatically completed upon the patient inputting a specific number of correct alpha-numeric characters. For example, referring to the memory test method 34 of FIG. 6C, if the patient inputs the characters “hou” and a correct response is “house,” the method automatically completes the entry for the patient and the patient is awarded one point. The automatic completion occurs regardless of whether the particular embodiment of the method uses a virtual keyboard, physical keyboard or both.

Also, in some embodiments, each of the patient's inputs is recorded and the various pieces of the test displayed are recorded so that a health care professional, such as a physician, can subsequently review the test as it was taken by the patient. For example, the attention test character layout is recorded, the timing of each and every patient input, and the total time to complete the test are all recorded for subsequent physician review.

The computer implemented method and system for cognitive testing, specifically for dementia-related afflictions, such as Alzheimer's disease is administered on a computer workstation in an interactive, web-based format and screens the cognitive abilities of the patient. The method includes evaluation of six areas of cognitive domain including visuospatial skills, executive function, memory, verbal fluency, attention, and orientation, all of which are essential for indications of dementia, including Alzheimer's disease and other cognitive afflictions. The method does not require administration by a trained professional, but rather, is administered in a web browser either from a server to the workstation via the Internet or locally at the workstation. Furthermore, the method compiles an overall test score indicating the cognitive function of the patient.

The foregoing description of preferred embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A computer implemented method for testing a patient's cognitive function including visuospatial skills and executive function, the method comprising:
(a) displaying a message regarding the proper shape of a clock face,
(b) receiving input from the patient indicating a choice in response to the message regarding the proper shape of the clock face,
(c) displaying a message regarding the proper positioning of clock hands to indicate a specific time of day,
(d) receiving input from the patient indicating a selection of position of clock hands in response to the message regarding the proper positioning of clock hands, and
(e) evaluating the patient's visuospatial skills and executive function based at least on the input received.

2. The method of claim 1 further comprising:
before step (b), displaying a plurality of shapes of a clock face at least one of which is a circle.

3. The method of claim 1 further comprising:
displaying a plurality of numbers including at least the numbers three, six, nine and twelve,
displaying a message regarding the proper placement of the plurality of numbers on the clock face, and
receiving input from the patient indicating a placement of the plurality of numbers in response to the message regarding proper placement of the numbers.

4. The method of claim 1 further comprising:
before step (d), displaying a plurality of positions of clock hands.

5. The method of claim 2 further comprising:
displaying a plurality of numbers including at least the numbers three, six, nine and twelve,
displaying a message regarding the proper placement of the plurality of numbers on the clock face,
receiving input from the patient indicating a placement of the plurality of numbers in response to the message regarding proper placement of the numbers, and
before step (d), displaying a plurality of positions of clock hands.

6. The method of claim 1 further comprising:
executing a software algorithm for generating workstation instructions on a computer server remote to a computer workstation;
receiving the workstation instructions at the computer workstation; and
executing the workstation instructions on the computer workstation to perform at least method steps (a) through (d).

7. The method of claim 1 wherein at least the displaying of steps (a) and (c) are performed using a web browser interface.

8. The method of claim 1 wherein steps (a) through (e) are performed by a computer workstation with no interaction from a human test administrator.

9. A computer implemented method for cognitive testing of a patient comprising:
(a) administering a clock drawing test including:
displaying a message regarding the proper shape of a clock face,
receiving input from the patient indicating a choice in response to the message regarding the proper shape of the clock face,
displaying a message regarding the proper positioning of clock hands to indicate a specific time of day,
receiving input from the patient indicating a selection of position of clock hands in response to the message regarding the proper positioning of clock hands, and
evaluating the patient's visuospatial skills and executive function based on the input received;
(b) administering a memory test including evaluating the patient's memory function;
(c) administering a verbal fluency test including evaluating the patient's verbal fluency;
(d) administering an attention test including evaluating the patient's attention function; and
(e) administering an orientation test including evaluating the patient's orientation function.

10. The method of claim 9 further comprising step (f) compiling the evaluations performed in one or more of steps (a) through (e) into an overall test score indicating cognitive function of the patient.

11. The method of claim 9 further comprising:
executing a software algorithm for generating workstation instructions on a computer server remote to a computer workstation;
receiving the workstation instructions at the computer workstation; and
executing the workstation instructions on the computer workstation to perform at least method steps (a) through (e).

12. The method of claim 9 wherein at least step (a) is performed using a web browser interface.

13. The method of claim 9 wherein steps (a) through (e) are performed by a computer workstation with no interaction from a human test administrator.

14. The method of claim 9 wherein administering the memory test further includes:
displaying a first message requesting the patient to memorize a memory item, discontinuing the display of the first message,
displaying a second message requesting the patient to recall the memory item,
receiving input from the patient indicating the patient's recall of the memory item, and
evaluating the patient's memory function based at least in part on the input received.

15. The method of claim 9 wherein administering the verbal fluency test further includes:
displaying a message requesting the patient to input a plurality of species names of a well-known genus,
receiving input from the patient indicating the patient's response to the message, and
evaluating the patient's verbal fluency based at least in part on the input received.

16. The method of claim 9 wherein administering the attention test further includes:
displaying a plurality of characters,
displaying a message regarding the characters,
receiving input from the patient indicating the patient's response to the message, and
evaluating the patient's attention function based at least in part on the input received.

17. The method of claim 9 wherein administering the orientation test further includes:
displaying at least one message requesting the patient to input at least one item selected from the group consisting of the current year, the current month, and the current day of the week,
receiving input from the patient in response to the at least one message, and
evaluating the patient's orientation function based at least in part on the input received.

18. The method of claim 9 wherein administering the clock drawing test further includes:
prior to receiving input from the patient indicating a choice regarding the proper shape of the clock face, displaying a plurality of shapes at least one of which is a circle,
displaying a plurality of numbers including at least the numbers three, six, nine and twelve,
displaying a message regarding the proper placement of the plurality of numbers on the clock face,
receiving input from the patient indicating a placement of the plurality of numbers in response to the message regarding proper placement of the numbers, and
prior to receiving input from the patient indicating a selection in response to the message regarding the proper positioning of clock hands, displaying a plurality of positions of clock hands.

19. The method of claim 17 wherein:
(b) administering the memory test includes:
displaying a message requesting the patient to commit a plurality of words to memory,
displaying the plurality of words,
discontinuing the display of the plurality of words,
displaying a message requesting the patient to recall the plurality of words previously displayed,
receiving input from the patient indicating the patient's recall of the plurality of words previously displayed, and
evaluating the patient's memory function based at least in part on the input received;
(c) administering a verbal fluency test includes:
displaying a message requesting the patient to input a plurality of species names of a well-known genus,
receiving input from the patient indicating the plurality of species names, and
evaluating the patient's verbal fluency based at least in part on the input received;
(d) administering an attention test includes:
displaying a plurality of characters arranged in a plurality of orientations and positions including a plurality of a predetermined character,
displaying a message regarding the predetermined character,
receiving input from the patient identifying a choice in response to the message, and
evaluating the patient's attention function based at least in part on the input received; and
(e) administering an orientation test includes:
displaying at least one message requesting the patient to input the current year, month, and day of the week,
receiving input from the patient in response to the at least one message, and
evaluating the patient's orientation function based at least in part on the input received.

20. The method of claim 10 wherein the overall test score indicates a level of cognitive impairment when the overall test score falls into a severe cognitive impairment range, a moderate to severe cognitive impairment range, a mild to moderate cognitive impairment range, an early cognitive impairment range, or a normal cognitive range.

21. The method of claim 9 further comprising displaying a welcome page before administering the clock drawing test including displaying at least one message regarding the patient and receiving input from the patient indicating a response to the message.

22. The method of claim 10 further comprising storing the overall test score for analysis based on comparison with other patients' overall test scores.

23. The method of claim 15 wherein the well-known genus includes animals and the plurality of species names includes everyday names of animals.

24. The method of claim 14 wherein the memory item is a word.

25. The method of claim 9 further comprising choosing one or more multimedia presentations from a database of multimedia presentations based at least in part on one or more of the evaluations performed in steps (a) through (e) and communicating the one or more multimedia presentations to the patient.

26. The method of claim 9 further comprising compiling the evaluations performed in one or more of steps (a) through (e) into one or more domain specific cognitive patterns.

27. The method of claim 26 further comprising choosing one or more multimedia presentations from a database of multimedia presentations based at least in part on the one or more domain specific cognitive patterns and communicating the one or more multimedia presentations to the patient.

28. The method of claim 9 wherein one or more of steps (a) through (e) includes receiving input from the patient via a virtual keyboard.

29. The method of claim 9 wherein one or more of steps (a) through (e) includes receiving alpha-numeric input from the patient and automatically completing one or more character strings based at least in part on a potential input database and the received alpha-numeric input.

30. The method of claim 9 wherein administering the attention test further includes:
displaying a plurality of months,
displaying a message regarding the plurality of months,
receiving input from the patient indicating the patient's response to the message, and
evaluating the patient's attention function based at least in part on the input received.

31. The method of claim 9 wherein the message requests the patient to input five months starting with a specific month and moving backward on the calendar.

32. A cognitive testing computer system comprising:
(a) means for administering a clock drawing test including:
means for displaying a message regarding the proper shape of a clock face,
means for receiving input from the patient indicating a choice in response to the message regarding the proper shape of the clock face,
means for displaying a message regarding the proper positioning of clock hands to indicate a specific time of day,
means for receiving input from the patient indicating a selection of position of clock hands in response to the message regarding the proper positioning of clock hands, and
means for evaluating the patient's visuospatial skills and executive function based on the input received;
(b) means for administering a memory test including evaluating the patient's memory function;
(c) means for administering a verbal fluency test including evaluating the patient's verbal fluency;
(d) means for administering an attention test including evaluating the patient's attention function;
(e) means for administering an orientation test including evaluating the patient's orientation function; and
(f) means for compiling the evaluations performed in one or more of (a) through (e) into an overall test score indicating cognitive function of the patient.
